(19) United States
(12) Patent Application Publication
(10) Pub. No.: US 2022/0267710 A1
(43) Pub. Date: Aug. 25, 2022

(54) CELL COLLECTING APPARATUS, CELL COLLECTING METHOD, AND COMPUTER-READABLE MEDIUM

(71) Applicant: OLYMPUS CORPORATION, Tokyo (JP)

(72) Inventors: Elena SHIMOJI, Tokyo (JP); Takuma DEZAWA, Tokyo (JP); Yoko KAMATO, Tokyo (JP)

(73) Assignee: OLYMPUS CORPORATION, Tokyo (JP)

(21) Appl. No.: 17/582,244

(22) Filed: Jan. 24, 2022

(30) Foreign Application Priority Data
Feb. 19, 2021 (JP) 2021-025421

(51) Int. Cl. C12M 1/34; C12M 1/36; C12M 1/00

(52) U.S. Cl. CPC C12M 41/44; C12M 41/48; C12M 29/00; C12M 41/00; C12M 41/36

(57) ABSTRACT

A cell collecting apparatus includes: a culturing device having a culture vessel and a collecting channel; and a control device. The culturing device cultures cells in a state where the cells are suspended in a liquid medium stored in the culture vessel. The control device controls the culturing device such that a suspension height, which is a height at which the target cell is suspended in the liquid medium, approaches a collection height, which is a height of an inlet of the collecting channel, the inlet configured to be used for cell collection from inside the culture vessel.

### CROSS REFERENCE TO RELATED APPLICATIONS

This application is based upon and claims the benefit of priority of the prior Japanese Patent Application No. 2021-025421, filed Feb. 19, 2021, the entire contents of which are incorporated herein by reference.

### BACKGROUND OF THE INVENTION

### Field of the Invention

The present disclosure relates to a cell collecting apparatus, a cell collecting method, and a computer-readable medium for collecting cells cultured by using suspension culture.

### Description of the Related Art

For promoting drug discovery and regenerative medicine using pluripotent stem cells, it is essential to use a culture technique of stably supplying a large amount of cells while maintaining a certain level of quality. For this reason, in recent years, suspension culture capable of culturing a larger amount of cells at a time than monolayer culture has attracted attention.

In addition, when cultured cells are used for clinical application, it is necessary to sort the cultured cells. For example, in the case of culturing stem cells, it is required to selectively collect a cell of interest, which is differentiated from a stem cell. Such a sorting technique in suspension culture is described in JP 2019-162093 A, for example. According to the technique described in JP 2019-162093 A, it is possible to separate and collect post-differentiation hemocyte cells from pre-differentiation stem cells constituting a cell mass.

### SUMMARY OF THE INVENTION

A cell collecting apparatus according to one aspect of the present invention includes: a culturing device including a culture vessel and a collecting channel, the culturing device configured to culture cells in a state where the cells are suspended in a liquid medium stored in the culture vessel; and a control device configured to control the culturing device such that a suspension height, which is a height at which a target cell to be collected among the cells is suspended in the liquid medium, approaches a collection height, which is a height of an inlet of the collecting channel, the inlet configured to be used for collecting the cell from inside the culture vessel.

A cell collecting method according to one aspect of the present invention includes: culturing cells by using a culturing device in a state where the cells are suspended in a liquid medium stored in a culture vessel; and controlling the culturing device by a control device such that a suspension height, which is a height at which a target cell to be collected among the cells is suspended in the liquid medium, approaches a collection height, which is a height of an inlet of a collecting channel, the inlet configured to be used for collecting the cell from inside the culture vessel.

A computer-readable medium according to one aspect of the present invention is a non-transitory computer-readable medium storing a program causing a computer to execute a process of controlling a culturing device configured to culture cells in a state where the cells are suspended in a liquid medium stored in the culture vessel such that a suspension height, which is a height at which a target cell to be collected among the cells is suspended in the liquid medium, approaches a collection height, which is a height of an inlet of a collecting channel, the inlet configured to be used for collecting the cell from inside the culture vessel.

### BRIEF DESCRIPTION OF THE DRAWINGS

FIG. 1 is a schematic configuration diagram illustrating an overall configuration of a cell collecting apparatus 1 according to a first embodiment;

FIG. 2 is a diagram illustrating a configuration of a culturing device 100 according to the first embodiment;

FIG. 3 is a diagram illustrating a configuration of a control device 100 according to the first embodiment;

FIG. 4 is a flowchart illustrating an example of a cell collecting process according to the first embodiment;

FIG. 5 is a flowchart illustrating an example of a flow velocity control process;

FIG. 6 is a flowchart illustrating another example of the flow velocity control process;

FIG. 7 is a flowchart illustrating still another example of the flow velocity control process;

FIG. 8 is a diagram illustrating a configuration of a culturing device 200 according to a second embodiment;

FIG. 9 is a diagram illustrating a structure for moving an inlet 221 of a collecting channel;

FIG. 10 is a flowchart illustrating an example of a cell collecting process according to the second embodiment;

FIG. 11 is a diagram illustrating a configuration of a culturing device 300 according to a third embodiment;

FIG. 12 is a flowchart illustrating an example of the cell collecting process according to the third embodiment;

FIG. 13 is a diagram illustrating a configuration of a culturing device 400 according to a fourth embodiment;

FIG. 14 is a diagram illustrating a configuration of a culturing device 500 according to a fifth embodiment;

FIG. 15 is a diagram illustrating a configuration of a culturing device 600 according to a sixth embodiment;

FIG. 16 is a diagram illustrating a method of confirming a suspension position of a cell;

FIG. 17 is a flowchart illustrating an example of a cell collecting process according to the sixth embodiment;

FIG. 18 is a diagram illustrating a configuration of a culturing device 700 according to a seventh embodiment;

FIG. 19 is a flowchart illustrating an example of a cell collecting process according to the seventh embodiment;

FIG. 20 is a diagram illustrating a configuration of a culturing device 800 according to an eighth embodiment; and

FIG. 21 is a flowchart illustrating an example of a cell collecting process according to the eighth embodiment.

### DESCRIPTION OF THE EMBODIMENTS

According to JP 2019-162093 A, a target cell is collected using a difference in suspension positions caused by a difference in size of a cell (or a cell mass). However, since this technique adopts a configuration of collecting a target cell from a liquid medium overflowed from a culture vessel, it is a premise of collection that the target cell is suspended at a position higher than other cells. For this reason, even with the technique described in JP 2019-162093 A, it is difficult to separate and collect an arbitrary cell from other cells, and thus, the cells that can be collected would be substantially limited.

In addition, whether it is possible to selectively collect a specific cell will not be determined by the cell itself, and depends on the relationship with other cells in the culture vessel. Therefore, when unknown cells are included in the culture environment, it is not always possible to appropriately collect only the cell of interest.

Hereinafter, embodiments of the present invention will be described.

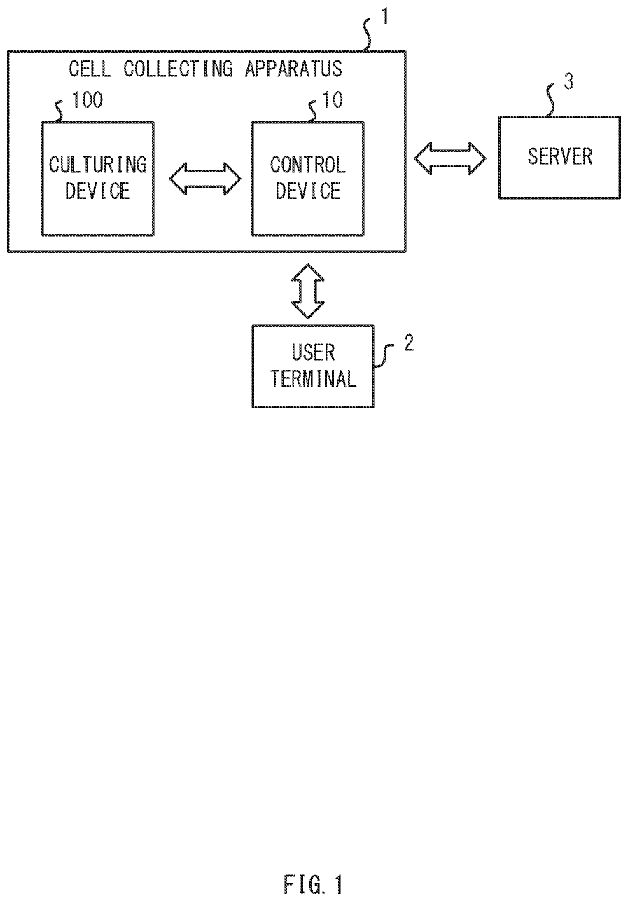

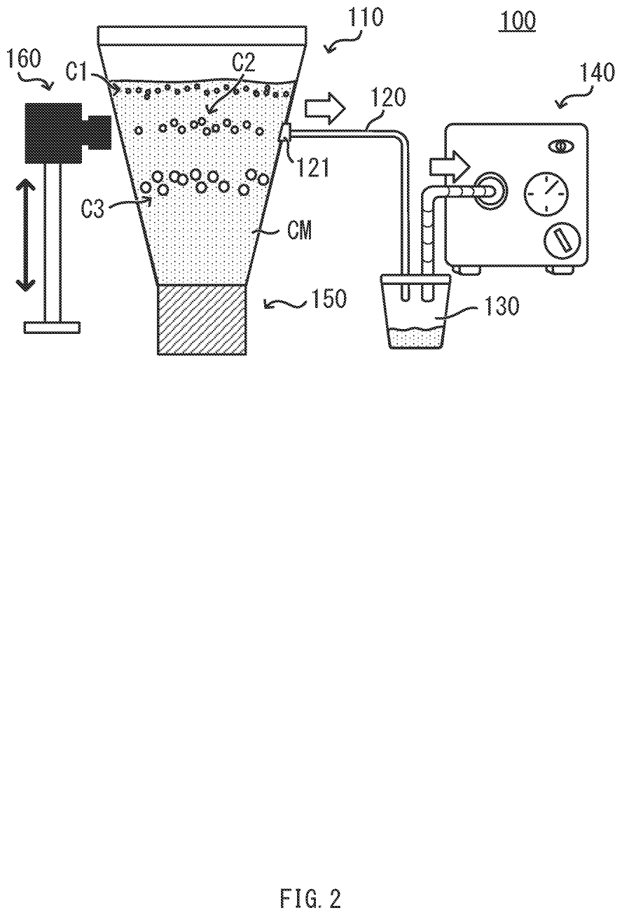

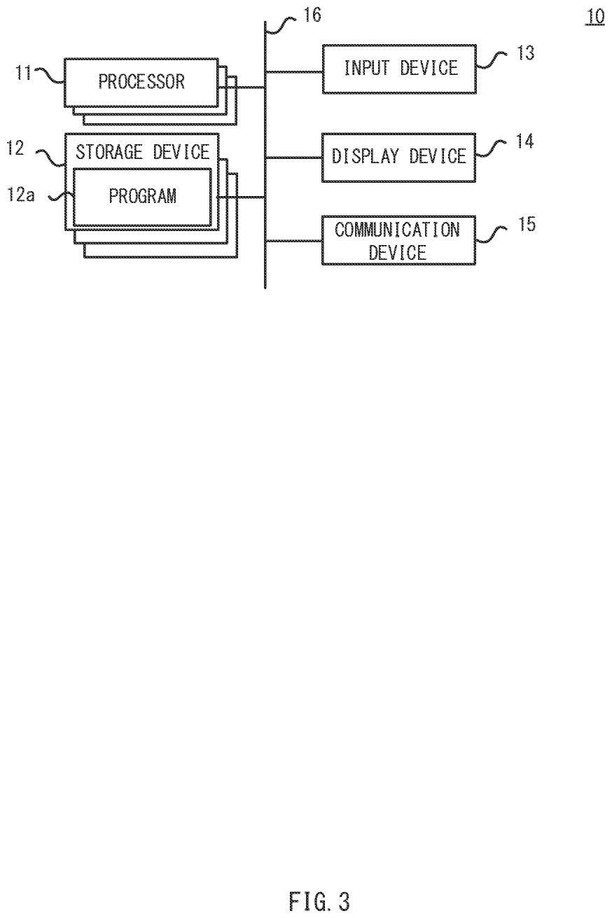

FIG. 1 is a schematic configuration diagram illustrating an overall configuration of a cell collecting apparatus 1 according to the present embodiment. FIG. 2 is a diagram illustrating a configuration of a culturing device 100 according to the present embodiment. FIG. 3 is a diagram illustrating a configuration of a control device 10 according to the present embodiment. Hereinafter, the configuration of the cell collecting apparatus 1 according to the present embodiment will be described with reference to FIGS. 1 to 3.

The cell collecting apparatus 1 is an apparatus that separates and collects a cell to be collected (hereinafter, referred to as a target cell) from cells suspended in a liquid medium, that is, an apparatus that sorts cells suspended in a liquid medium. The target cell is not particularly limited, but may be a predetermined cell differentiated from a stem cell to a specialized type of cell, for example. As illustrated in FIG. 1, the cell collecting apparatus 1 includes the culturing device 100 that cultures cells and the control device 10 that controls the culturing device 100.

The cell collecting apparatus 1 may communicate with a user terminal 2, or may collect cells following an instruction from the user terminal 2. In addition, the cell collecting apparatus 1 may notify the user terminal 2 of the collection progress, the collection result, and the like. Furthermore, the cell collecting apparatus 1 may communicate with a server 3, and may acquire information necessary for collecting cells from the server 3.

The culturing device 100 is a device that cultures cells (for example, cell C1, cell C2, and cell C3) in a state of being suspended in a liquid medium CM stored in a culture vessel 110 and collects a target cell (for example, cell C2) from the culture vessel 110 to a collecting vessel 130 via a collecting channel formed in a collecting nozzle 120. More specifically, as illustrated in FIG. 2, the culturing device 100 includes the culture vessel 110, the collecting nozzle 120, the collecting vessel 130, a suction device 140, a suspension device 150, and a camera 160.

The culture vessel 110 stores the liquid medium CM in which cells are suspended. In the culture vessel 110, an upward flow is generated in the liquid medium CM by the effects of the suspension device 150 to be described below. With this upward flow, the cells are cultured in a state of being suspended in the liquid medium CM in the culture vessel 110. The culture vessel 110 may be a bioreactor, for example, and may be provided with a channel for replacement of a liquid medium (collection and supply of a medium) and various sensors for measuring pH, temperature, and the like.

The culture vessel 110 has a truncated cone shape. More specifically, the shape is a truncated cone shape having an upper base diameter larger than a lower base diameter, that is, a shape referred to as an inverted truncated cone shape. Note that the shape of the culture vessel 110 is not limited to the inverted truncated cone shape. The culture vessel 110 only needs to be able to generate an upward flow having a flow velocity difference in the height direction in the culture vessel 110, in combination with the suspension device 150. That is, the shape of the culture vessel 110 is only required to be able to provide a flow velocity difference of upward flow in the height direction, and is desirably a shape having a cross-sectional area difference in the height direction. It is particularly desirable to have a shape having a larger cross-sectional area with a greater height, that is, a shape in which the flow velocity can be reduced as the height increases, such as an inverted truncated cone shape. However, for example, the shape of the culture vessel 110 may be designed as described in JP 2018-201423 A, and the upward flow may have a discontinuous velocity in the height direction in the medium.

The collecting nozzle 120 is a nozzle having a channel (collecting channel) for guiding the cells collected from the culture vessel 110 to the collecting vessel 130. An inlet 121 of the collecting channel is used for collecting cells from the culture vessel 110. The inlet 121 is formed at a known position or height on a side surface of the culture vessel 110. Hereinafter, the height of the inlet 121 is referred to as a collection height in the sense of a height when the cell in the culture vessel 110 is collected to the outside of the culture vessel 110.

Although FIG. 2 illustrates one collecting nozzle 120, the number of collecting nozzles 120 is not necessarily limited to one. For example, a plurality of the collecting nozzles 120 having a same collection height may extend from the side surface of the culture vessel 110. That is, the inlets 121 of the plurality of collecting channels may be aligned in a circumferential direction on the side surface of the culture vessel 110.

The collecting vessel 130 is a vessel that stores the cells collected from the culture vessel 110. The collecting vessel 130 may have a filter that makes further strict distinction of the cells selectively collected from the culture vessel 110 by the size of the cells and other characteristics. That is, the cell collecting apparatus 1 may separate the target cell from other objects and collect the target cell by the physical structure of the filter provided in the collecting vessel 130, in addition to the control of the culturing device 100 by the control device 10 described below. The filter may be a filter such as a mesh filter or a hollow fiber filter capable of distinguishing the target cell from other objects by size or other characteristics.

The suction device 140 is a device for generating suction force in the inlet 121 of the collecting channel. The suction device 140 only needs to be able to generate suction force sufficient to draw the cells from the collecting vessel 130 to the collecting nozzle 120. The suction device 140 may be a suction pump that applies a negative pressure to the collecting vessel 130 by sucking air in the collecting vessel 130, for example. In addition, the suction device 140 may be, for example, a liquid feeding pump such as a peristaltic pump, and may act on the collecting nozzle 120 between the culture vessel 110 and the collecting vessel 130 in a state where one and the other ends of the collecting nozzle 120 are immersed in the liquid (medium) contained in the culture vessel 110 and the collecting vessel 130, respectively.

The suction device 140 is controlled by the control device 10. For example, the suction device 140 may start operation upon reception of a signal from the control device 10, and may stop operation upon reception of a signal from the control device 10. The suction device 140 operates to generate suction force in the inlet 121 of the collecting channel. By the suction force generated in the suction device 140 and the inlet 121, the cell suspended in the liquid medium CM is sucked from the culture vessel 110, and then sucked cell is output to the collecting vessel 130 via the collecting channel.

The suspension device 150 is a device that generates an upward flow in the liquid medium CM in the culture vessel 110 to suspend the cells. More specifically, the suspension device 150 is, for example, a device that ejects a helical flow of the liquid medium CM from the lower side to the upper side of the culture vessel 110 as described in JP 2019-162093 A. However, the suspension device 150 is not limited to the device that ejects the helical flow, and the method of generating the upward flow by the suspension device 150 is not particularly limited.

The suspension device 150 is controlled by the control device 10. For example, the suspension device 150 may start operation upon reception of a signal from the control device 10, and may stop operation upon reception of a signal from the control device 10. Furthermore, the suspension device 150 may change the ejection amount of the helical flow upon reception of a signal from the control device 10, for example. With operation of the suspension device 150, an upward flow occurs in the liquid medium CM in the culture vessel 110 so as to allow the cells to be suspended in the liquid medium CM.

When used in combination with the culture vessel 110 having an inverted truncated cone shape having greater cross-sectional area at a greater height, the suspension device 150 generates, in the liquid medium CM in the culture vessel 110, an upward flow having a flow velocity difference in the height direction. More particularly, the greater the height, the lower the flow velocity in the upward flow generated.

In the culture vessel 110, the cell is suspended at a position where the flow velocity of the upward flow generated by the suspension device 150 and the sedimentation velocity of the cell are balanced. As described above, the flow velocity of the upward flow in culture vessel 110 varies in the height direction, and thus, the greater the height, the lower the flow velocity. For this reason, the cell is suspended at a height corresponding to the sedimentation velocity, and thus, the lower the sedimentation velocity, the higher the position at which the cell is suspended.

The terminal sedimentation velocity of a cell can be estimated by the Stokes' law. According to the Stokes' law, the terminal sedimentation velocity depends on the diameter and density of the cells as well as the density and viscosity of the liquid medium CM. In particular, the terminal sedimentation velocity is proportional to the square of the diameter of the cell, and thus, dominantly affected by the size of the cell. Therefore, in the culturing device 100, the cell is suspended at a height corresponding to the size of the cell in accordance with the combination of the culture vessel 110 and the suspension device 150, and specifically, the smaller the size of the cell, the higher the position at which the cell is suspended.

The camera 160 is an imaging device that captures an image of a target cell, and includes an imaging element. The imaging element is, for example, a two-dimensional image sensor such as a charge coupled device (CCD) image sensor or a complementary metal oxide semiconductor (CMOS) image sensor. Note that the camera 160 only needs to have resolution sufficient to identify the cells suspended in the liquid medium CM in the culture vessel 110, and desirably has resolution capable of identifying types, cell mutations, differentiation states, and the like of various cells (cell C1, cell C2, and cell C3) present in the culture vessel 110. Furthermore, the camera 160 can appropriately change the imaging position. This makes it possible to image cells suspended at various heights.

The camera 160 is controlled by the control device 10. The camera 160 may start imaging operation upon reception of a signal from the control device 10, for example. The image acquired by the camera 160 is output to the control device 10.

The control device 10 is a device that controls the culturing device 100 in order to collect the target cell. The control device 10 controls the culturing device 100 so that the height at which the target cell is suspended in the liquid medium CM (hereinafter, referred to as a suspension height) approaches the height of the inlet 121 of the collecting channel (collection height). Specifically, the control device 10 controls the culturing device 100 such that the suspension height approaches the collection height based on a result of object detection on the image acquired by the camera 160.

In order to execute the above control, the control device 10 may further perform object detection on the image acquired by the camera 160. For object detection, it is allowable to use a trained model obtained by deep learning. In this case, it is desirable that the trained model has learned at least the target cell. It is desirable that the trained model has further learned cells other than the target cell and objects other than the cells. In addition, object detection may be performed using a trained model obtained by machine learning other than deep learning, and an algorithm of detecting a target cell based on a known feature such as a shape of the target cell may be used for object detection.

The object detection specifies the image in which the target cell is detected, and further specifies the position and size of the target cell in the image. As a result, the position and size of the target cell are specified. That is, the result of the object detection includes at least position information regarding the target cell and size information regarding the target cell.

Note that the object detection may be performed by a device other than the control device 10, for example, the server 3. The control device 10 may transmit the image acquired by the camera 160 to the server 3 and receive from the server 3 the result of the object detection executed by the server 3.

In the cell collecting apparatus 1, the control device 10 controls the flow velocity of the upward flow so that the suspension height approaches the collection height based on the result of the object detection. This corresponds to the control of the suspension height. When the suspension height sufficiently approaches the collection height, the control device 10 activates the suction device 140 to collect the target cell suspended near the inlet 121.

Note that the control device 10 is only required to include one or more processors and one or more pieces of non-transitory computer-readable media. More specifically, for example, as illustrated in FIG. 3, the control device 10 may include one or more processors 11, one or more storage devices 12, an input device 13, a display device 14, and a communication device 15, and these devices may be interconnected via a bus 16.

Each of the one or more processors 11 is a piece of hardware including, for example, a central processing unit (CPU), a graphics processing unit (GPU), a digital signal processor (DSP), and the like, and executes a program 12a stored in the one or more storage devices 12 to perform programmed processing. In addition, the one or more processors 11 may include an application specific integrated circuit (ASIC), a field-programmable gate array (FPGA), and the like.

Each of the one or more storage devices 12 is a non-transitory computer-readable medium and includes, for example, one or a plurality of pieces of arbitrary semiconductor memory, and may further include one or more other storage devices. The semiconductor memory includes, for example, volatile memory such as random access memory (RAM), and nonvolatile memory such as read only memory (ROM), programmable ROM, and a flash memory. The RAM may include, for example, dynamic random access memory (DRAM), static random access memory (SRAM), and the like. The other storage devices may include, for example, a magnetic storage device including a magnetic disk, an optical storage device including an optical disk, and the like.

The input device 13 is a device directly operated by a user, and examples of this include a keyboard, a mouse, and a touch panel. The display device 14 is, for example, a liquid crystal display, an organic EL display, a cathode ray tube (CRT) display, or the like. The display may include a built-in touch panel. The communication device 15 may be a wired communication module or a wireless communication module.

Note that the configuration illustrated in FIG. 3 is an example of a hardware configuration of the control device 10, and the control device 10 is not limited to this configuration. The control device 10 may be a general-purpose device or a dedicated device.

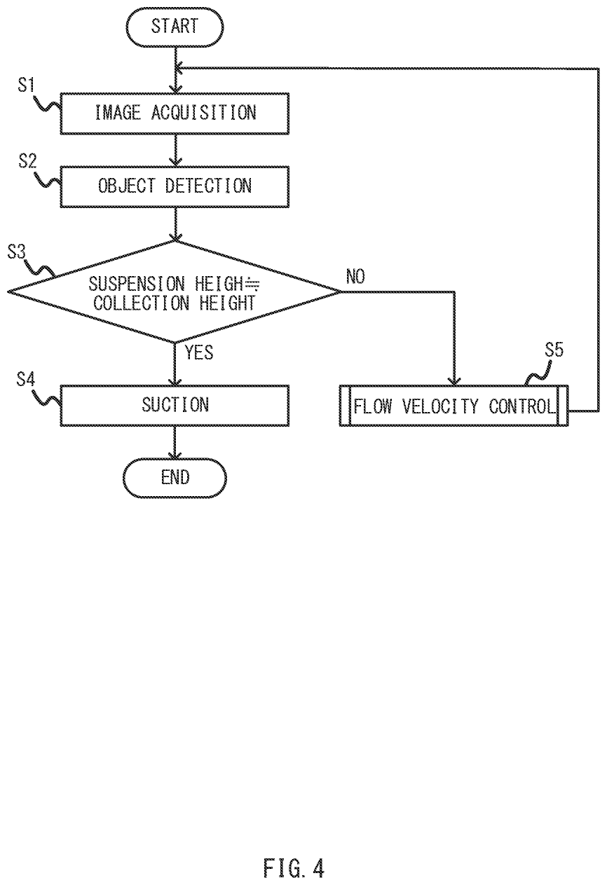

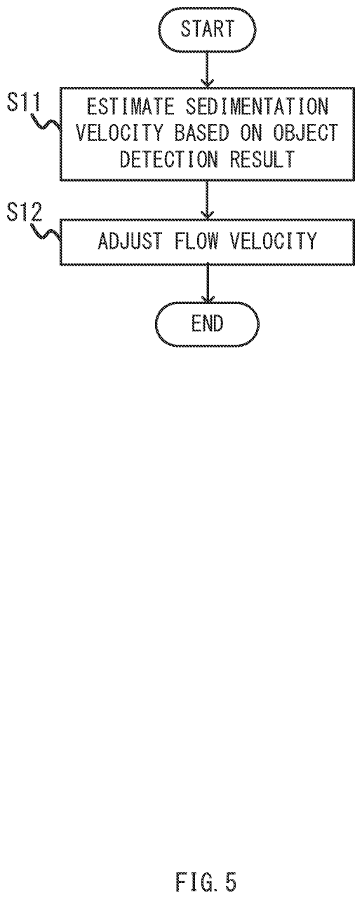

FIG. 4 is a flowchart illustrating an example of a cell collecting process according to the present embodiment. FIG. 5 is a flowchart illustrating an example of the flow velocity control process. Hereinafter, a cell collecting method performed using the cell collecting apparatus 1 will be specifically described with reference to FIGS. 4 and 5.

The cell collecting process illustrated in FIG. 4 is started in accordance with a user's start instruction or a set schedule, for example, upon execution of a cell collecting program stored in the storage device 12 by the processor 11 of the control device 10.

When the cell collecting process is started, the cell collecting apparatus 1 performs imaging of a target cell and acquires an image (step S1). In step S1, the control device 10 controls the camera 160 so that the camera 160 acquires, for example, images a plurality of times while changing the position in the height direction and thereby captures an image of the target cell. The target cell may be determined in advance, or may be selected by the user using the acquired image.

Next, the cell collecting apparatus 1 performs object detection on the image acquired in step S1 (step S2). In step S2, the control device 10 performs object detection using the trained model that has learned the target cell. This specifies the position and size of the target cell.

Thereafter, the cell collecting apparatus 1 determines whether the suspension height is sufficiently close to the collection height (step S3). In the cell collecting apparatus 1, the collection height is known in advance. Therefore, in step S3, the control device 10 specifies the suspension height from the position information regarding the target cell included in the result of the object detection in step S2, compares the specified suspension height with the known collection height, and then determines whether the difference of the heights is within a threshold.

When it is determined in step S3 that the suspension height is sufficiently close to the collection height (step S3 YES), the cell collecting apparatus 1 sucks the target cell (step S4). In step S4, the control device 10 controls the suction device 140 to generate suction force in the inlet 121 so as to suck the target cell suspended in the vicinity of the inlet 121 into the collecting channel.

In contrast, when it is determined in step S3 that the suspension height is not sufficiently close to the collection height (step S3 NO), the cell collecting apparatus 1 performs a flow velocity control process illustrated in FIG. 5 (step S5). In step S5, the control device 10 first estimates the sedimentation velocity based on the result of the object detection in step S2 (step S11).

In step S11, the control device 10 may estimate the sedimentation velocity using the Stokes' law. Specifically, the control device 10 may estimate the diameter of the target cell from the size of the target cell obtained in step S2, and may estimate the sedimentation velocity using the estimated diameter. Note that a predetermined value may be used for other parameters (for example, density of cells, density and viscosity of liquid medium CM) included in the Stokes' law.

In step S11, the sedimentation velocity may be estimated using the position of the target cell instead of the size of the target cell obtained in step S2. Since the target cell is suspended at a position where the sedimentation velocity and the flow velocity of the upward flow are balanced. Accordingly, the flow velocity at each position of the upward flow is known, the sedimentation velocity can be estimated from the position of the target cell (suspension position).

When the sedimentation velocity is estimated, the control device 10 adjusts the flow velocity of the upward flow in accordance with the estimated sedimentation velocity (step S12). In step S12, the control device 10 controls the suspension device 150 so that the flow velocity of the upward flow at the height (collection height) of the inlet 121 approaches, desirably becomes equal to, the estimated sedimentation velocity.

Thereafter, the cell collecting apparatus 1 repeats the processes from step S1 to step S3 and step S5 until the suspension height sufficiently approaches the collection height by the flow velocity control. When the suspension height sufficiently approaches the collection height, the suction process in step S4 is executed to end the cell collecting process illustrated in FIG. 4.

According to the cell collecting apparatus 1, execution of the cell collecting process illustrated in FIG. 4 makes it possible to automatically select and collect the target cell from the plurality of objects suspended in the liquid medium. That is, it is possible to sort the cells. In particular, on the cell collecting apparatus 1, by adopting a configuration in which the target cell is collected from the inlet 121 of the collecting channel provided on the side surface of the culture vessel 110, and further changing the flow velocity of the upward flow, it is possible to select the cell suspended in the vicinity of the inlet 121. Therefore, according to the cell collecting apparatus 1, unlike the device described in JP 2019-162093 A in which target cell can be collected only when suspended at a position higher than other cells, it is possible to collect an arbitrary cell regardless of the relationship with other objects present in the liquid medium CM. In addition, even when an unknown object is suspended in the liquid medium CM, only the target cell can be automatically collected.

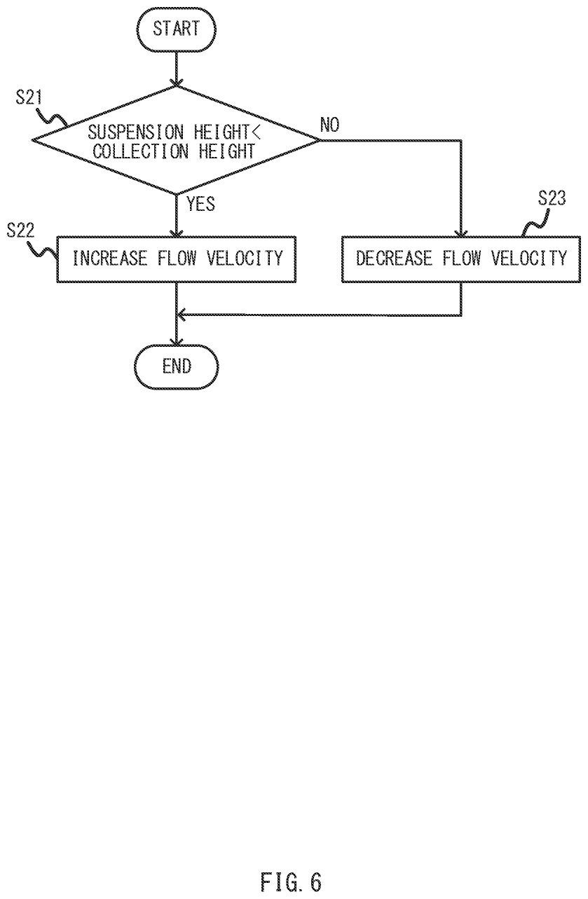

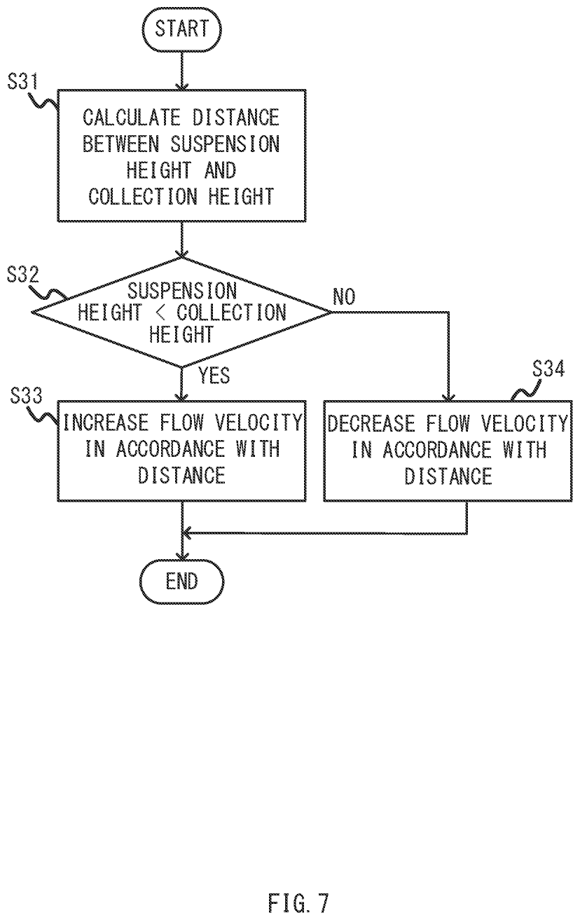

FIGS. 6 and 7 are flowcharts illustrating mutually different examples of the flow velocity control process. Although the above has described an example in which the control device 10 performs the flow velocity control illustrated in FIG. 5, the control device 10 may perform the flow velocity control illustrated in FIG. 6 or 7 instead of the flow velocity control illustrated in FIG. 5.

As illustrated in FIG. 6, the control device 10 may first compare the suspension height with the collection height in the flow velocity control in step S5 (step S21). When having determined that the suspension height is lower than the collection height (step S21 YES), the control device 10 may increase the flow velocity of the upward flow (step S22); when having determined that the suspension height is not lower than the collection height (step S21 NO), the control device may decrease the flow velocity of the upward flow (step S23). That is, unlike the flow velocity control illustrated in FIG. 5, the control device 10 may control the suspension device 150 so as to increase or decrease the flow velocity of the upward flow based on the comparison result between the suspension height and the collection height.

In the cell collecting apparatus 1, the control device 10 may perform feedback control so that the suspension position approaches the collecting position by repeating the flow velocity control illustrated in FIG. 6. According to the flow velocity control illustrated in FIG. 6, since the flow velocity is controlled based on the difference in height actually occurring between the suspension position and the collecting position, it is possible to align the suspension position and the collecting position without estimating the sedimentation velocity. Therefore, also by performing the flow velocity control illustrated in FIG. 6, the cell collecting apparatus 1 can collect an arbitrary cell and collect only the target cell similarly to the case of performing the flow velocity control illustrated in FIG. 5.

As illustrated in FIG. 7, the control device 10 may calculate a distance between the suspension height and the collection height before comparing the suspension height and the collection height (step S31). Thereafter, the control device 10 may compare the suspension height and the collection height (step S32). In a case where it is determined that the suspension height is lower than the collection height (step S32 YES), the control device 10 may increase the flow velocity of the upward flow in accordance with the distance calculated in step S31 (step S33). In a case where it is determined that the suspension height is not lower than the collection height (step S32 NO), the control device 10 may decrease the flow velocity of the upward flow in accordance with the distance calculated in step S31 (step S34). That is, the control device 10 may control the suspension device 150 so as to increase or decrease the flow velocity of the upward flow in accordance with the distance between the suspension height and the collection height based on the comparison result between the suspension height and the collection height. More specifically, it is allowable to set such that the greater the distance, the larger the amount of increase or decrease of the flow velocity of the upward flow.

In the cell collecting apparatus 1, the control device 10 may perform feedback control so that the suspension position approaches the collecting position by repeating the flow velocity control illustrated in FIG. 7. According to the flow velocity control illustrated in FIG. 7, since the flow velocity is controlled based on the difference in height actually occurring between the suspension position and the collecting position, it is possible to align the suspension position and the collecting position without estimating the sedimentation velocity. In addition, the flow velocity control illustrated in FIG. 7 adjusts the amount of increase or decrease in the flow velocity in accordance with the magnitude of the difference in height (that is, the distance), and thus, the suspension position and the collecting position can be aligned in a shorter time than in the case of performing the flow velocity control illustrated in FIG. 6.

Although the above is an example in which suction is immediately performed when it is determined that the suspension height is sufficiently close to the collection height (step S3 YES in FIG. 4), it is also allowable to provide a human confirmation process before suction. For example, when having determined that the suspension height is sufficiently close to the collection height in step S3, the control device 10 may monitor user's input of information and wait until an input is made. In this case, the user may visually check the culture vessel, determine the relationship between the suspension height and the collection height, and may input an instruction regarding the increase or decrease of the flow velocity to the control device 10, and the control device 10 may then finely adjust the flow velocity by controlling the suspension device 150. After the flow velocity is finely adjusted, the control device 10 may control the suction device 140 to collect the target cell.

### Second Embodiment

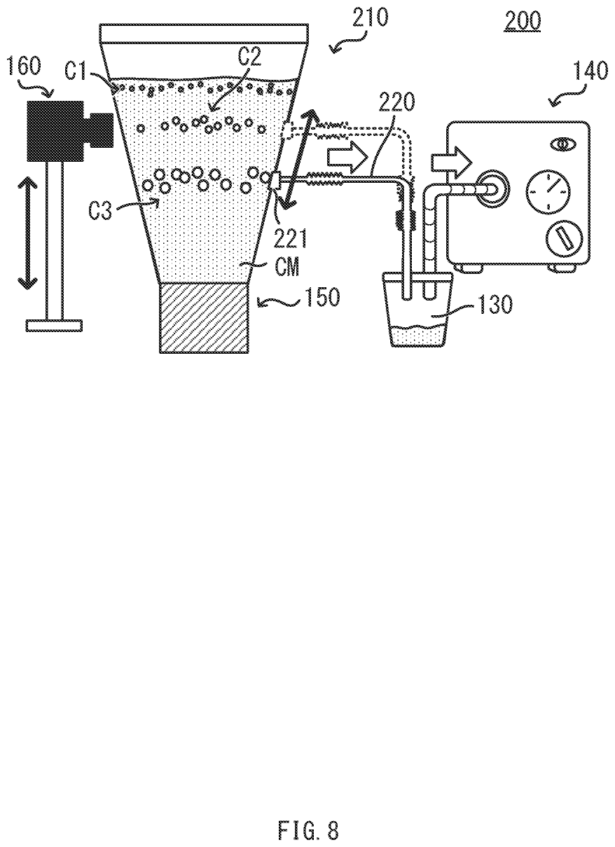

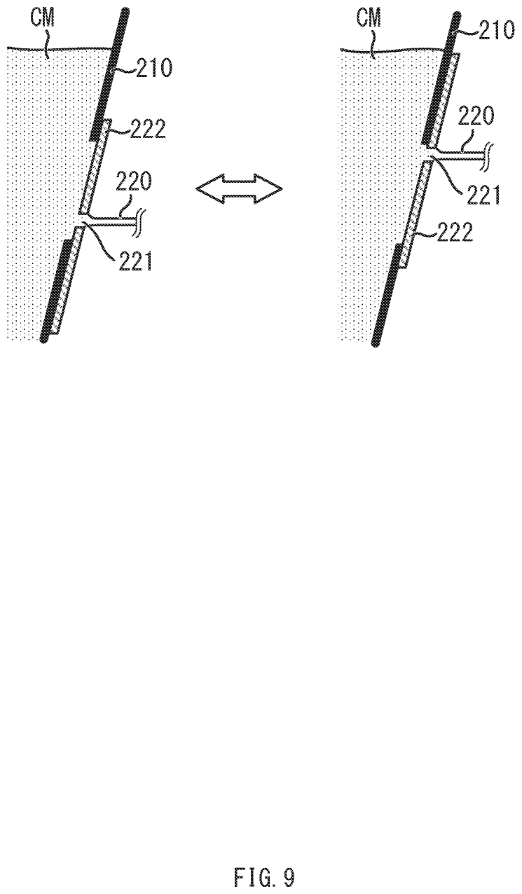

FIG. 8 is a diagram illustrating a configuration of a culturing device 200 according to the present embodiment. FIG. 9 is a diagram illustrating a structure for moving an inlet 221 of the collecting channel. Hereinafter, the configuration of the cell collecting apparatus according to the present embodiment (hereinafter, simply referred to as a cell collecting apparatus) will be described with reference to FIGS. 8 and 9.

The cell collecting apparatus is different from the cell collecting apparatus 1 according to the first embodiment in that the cell collecting apparatus includes the culturing device 200 illustrated in FIG. 8 instead of the culturing device 100. The cell collecting apparatus is similar to the cell collecting apparatus 1 in that the control device 10 is included.

The culturing device 200 is different from the culturing device 100 in that a collecting nozzle 220 has a stretchable structure as illustrated in FIG. 8 and that a slider 222 having a structure for moving the inlet 221 of the collecting nozzle 220 in the height direction is provided on a side surface of a culture vessel 210 as illustrated in FIG. 9. The slider 222 is an example of a height adjuster that adjusts the collection height, and the position of the slider 222 is controlled by the control device 10.

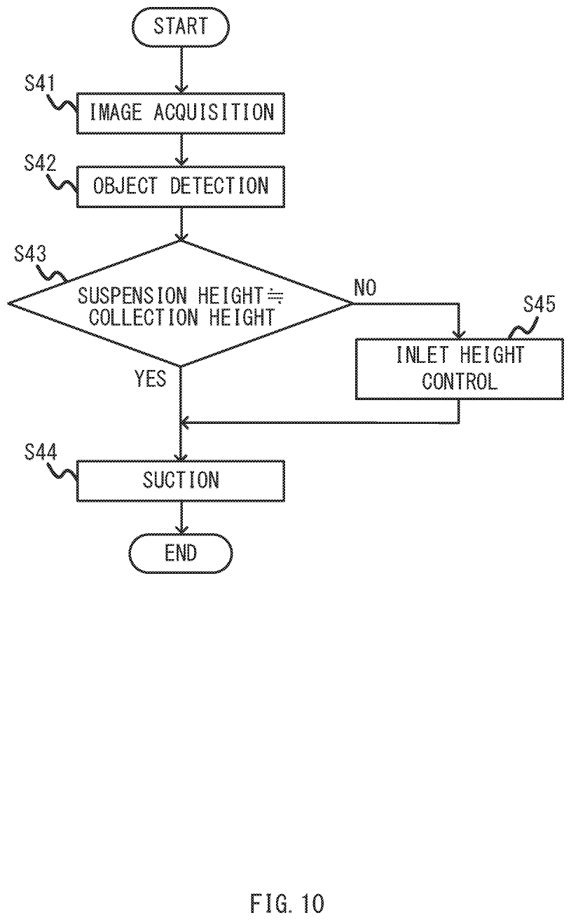

FIG. 10 is a flowchart illustrating an example of the cell collecting process according to the present embodiment. Hereinafter, a cell collecting method implemented by using the cell collecting apparatus according to the present embodiment will be specifically described with reference to FIG. 10.

The cell collecting process illustrated in FIG. 10 is started in accordance with a user's start instruction or a set schedule upon execution of the cell collecting program stored in the storage device 12 by the processor 11 of the control device 10.

When the cell collecting process is started, the cell collecting apparatus captures an image of a target cell to acquire the image (step S41), and performs object detection on the image acquired in step S41 (step S42). The processes in steps S41 and S42 are similar to the processes in steps S1 and S2 in FIG. 4, respectively.

Thereafter, the cell collecting apparatus determines whether the suspension height is sufficiently close to the collection height (step S43). In the cell collecting apparatus, the collection height is known because the height is determined by the position of the slider 222. Therefore, in step S43, the control device 10 specifies the suspension height from the position information regarding the target cell included in the result of the object detection in step S42, compares the specified suspension height with the known collection height, and then determines whether the difference of the heights is within a threshold.

When it is determined in step S43 that the suspension height is sufficiently close to the collection height (step S43 YES), the cell collecting apparatus sucks the target cell (step S44). The process in step S44 is similar to the process in step S4 in FIG. 4.

In contrast, when it is determined in step S43 that the suspension height is not sufficiently close to the collection height (step S43 NO), the cell collecting apparatus controls the height of the inlet 221 (step S45). In step S45, the control device 10 moves the slider 222 in the height direction to bring the collection height close to the specified suspension height based on the result of the object detection. More desirably, the control device 10 controls the collection height to match the suspension height. When the height control of the inlet 221 is completed, the cell collecting apparatus sucks the target cell (step S44). The process in step S44 is similar to the process in step S4 in FIG. 4.

As described above, similarly to the first embodiment, the control device 10 in the present embodiment also controls the culturing device 200 so that the suspension height approaches the collection height based on the result of object detection on the image acquired by the camera 160. Note that the present embodiment is different from the first embodiment in that the control device 10 controls the slider 222 being a height adjuster such that the suspension height approaches the collection height based on the result of object detection. That is, in contrast to the first embodiment in which the suspension height is brought close to the collection height by adjusting the suspension height, the second embodiment uses a method in which the suspension height is brought close to the collection height by adjusting the collection height. More specifically, the control device 10 controls the slider 222 so that the slider 222 moves the inlet 221 in a direction of approaching the suspension height based on the result of object detection.

Also with the cell collecting apparatus according to the present embodiment, by executing the cell collecting process illustrated in FIG. 10, it is possible to automatically select and collect the target cell from a plurality of objects suspended in the liquid medium, similarly to the cell collecting apparatus 1 according to the first embodiment. This embodiment is similar to the above embodiment in that an arbitrary cell can be collected and that only the target cell can be collected.

Although the first embodiment is an example in which the suspension height is adjusted to bring the suspension height close to the collection height and the second embodiment is an example in which the collection height is adjusted to bring the suspension height close to the collection height, it is also allowable to execute these adjustments in combination. That is, the suspension height may be brought close to the collection height by adjusting both the suspension height and the collection height.

### Third Embodiment

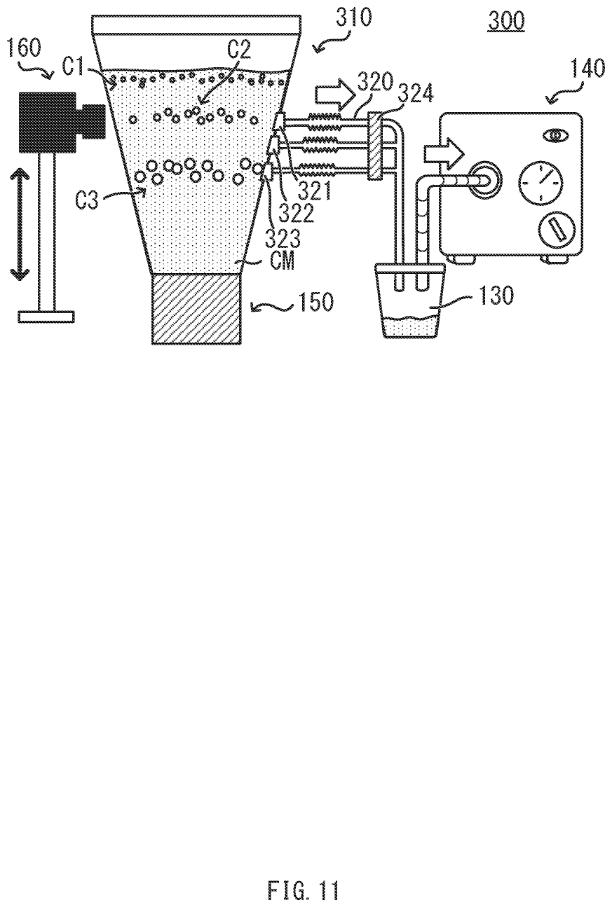

FIG. 11 is a diagram illustrating a configuration of a culturing device 300 according to the present embodiment. Hereinafter, the configuration of the cell collecting apparatus according to the present embodiment (hereinafter, simply referred to as a cell collecting apparatus) will be described with reference to FIG. 11.

The cell collecting apparatus is different from the cell collecting apparatus 1 according to the first embodiment in that the cell collecting apparatus includes the culturing device 300 illustrated in FIG. 11 instead of the culturing device 100. The cell collecting apparatus is similar to the cell collecting apparatus 1 in that the control device 10 is included.

As illustrated in FIG. 11, the culturing device 300 is different from the culturing device 100 in that a collecting nozzle 320 has a plurality of inlets (inlet 321, inlet 322, and inlet 323) facing a culture vessel 310 and having mutually different heights and that the collecting nozzle 320 includes a switching valve 324 that switches an inlet to be used for cell collection among the plurality of inlets. The switching valve 324 is an example of a height adjuster that adjusts the collection height, and opening and closing of the switching valve 324 is controlled by the control device 10.

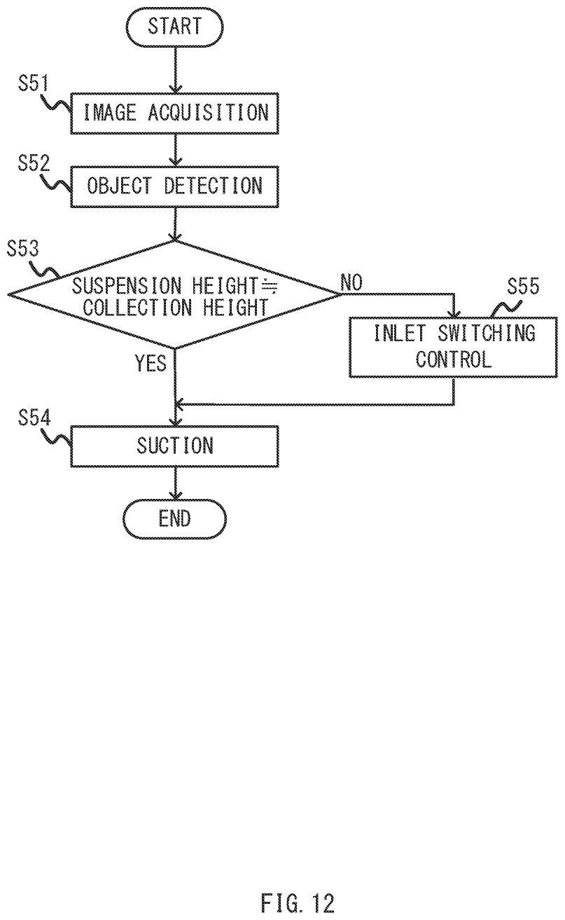

FIG. 12 is a flowchart illustrating an example of the cell collecting process according to the present embodiment. Hereinafter, a cell collecting method implemented by using the cell collecting apparatus according to the present embodiment will be specifically described with reference to FIG. 12.

The cell collecting process illustrated in FIG. 12 is started in accordance with a user's start instruction or a set schedule upon execution of the cell collecting program stored in the storage device 12 by the processor 11 of the control device 10.

When the cell collecting process is started, the cell collecting apparatus captures an image of a target cell to acquire the image (step S51), performs object detection on the image acquired in step S51 (step S52), and determines whether the suspension height is sufficiently close to the collection height (step S53). The processes from step S51 to step S53 are similar to the processes from step S41 to step S43 in FIG. 10, respectively.

When it is determined in step S53 that the suspension height is sufficiently close to the collection height (step S53 YES), the cell collecting apparatus sucks the target cell (step S54). The process in step S54 is similar to the process in step S44 in FIG. 10.

In contrast, when it is determined in step S53 that the suspension height is not sufficiently close to the collection height (step S53 NO), the cell collecting apparatus controls switching of the inlet used for cell collection (step S55). In step S55, based on the result of the object detection in step S52, the control device 10 controls the switching valve 324 so that the switching valve 324 switches the inlet to be used for cell collection to the inlet (for example, the inlet 321) closest to the suspension height among the plurality of inlets. After the end of the switching control of the inlet, the cell collecting apparatus sucks the target cell (step S54). The process in step S54 is similar to the process in step S44 in FIG. 10.

As described above, similarly to the second embodiment, the control device 10 in the present embodiment also controls the culturing device 300 so that the suspension height approaches the collection height based on the result of object detection on the image acquired by the camera 160. Furthermore, similarly to the second embodiment, the control device 10 controls the height adjuster so that the suspension height approaches the collection height based on the result of the object detection. That is, the suspension height is brought close to the collection height by adjusting the collection height. Note that the present embodiment is different from the second embodiment in that the control device 10 controls the switching valve 324 being a height adjuster such that the suspension height approaches the collection height based on the result of object detection. More specifically, based on the result of the object detection, the control device 10 controls the switching valve 324 so that the switching valve 324 switches the inlet to be used for cell collection to the inlet closest to the suspension height among the plurality of inlets.

Also with the cell collecting apparatus according to the present embodiment, by executing the cell collecting process illustrated in FIG. 12, it is possible to automatically select and collect the target cell from a plurality of objects suspended in the liquid medium, similarly to the cell collecting apparatus 1 according to the first embodiment and the cell collecting apparatus according to the second embodiment. This embodiment is similar to the above embodiment in that an arbitrary cell can be collected and that only the target cell can be collected.

Also in the present embodiment, the suspension height may be brought close to the collection height by adjusting both the suspension height and the collection height. This point is similar to that of the second embodiment.

### Fourth Embodiment

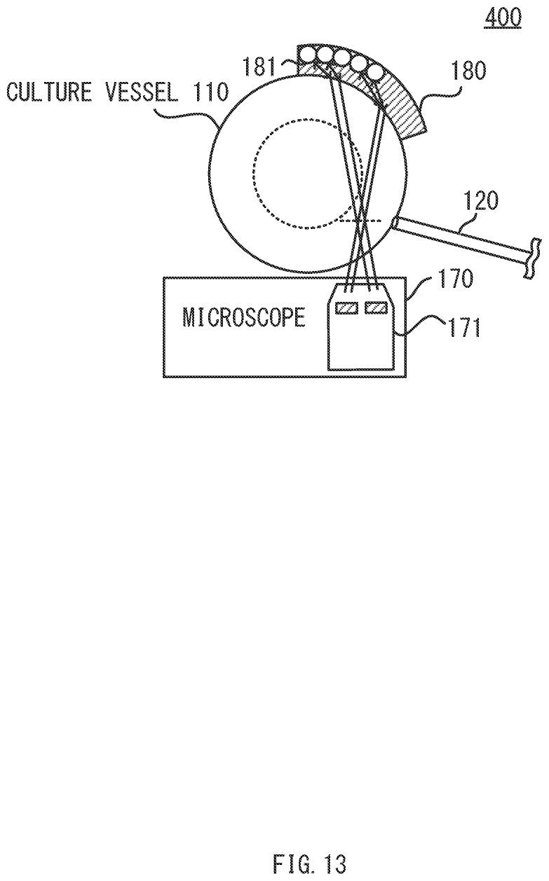

FIG. 13 is a diagram illustrating a configuration of a culturing device 400 according to the present embodiment. Hereinafter, the configuration of the cell collecting apparatus according to the present embodiment (hereinafter, simply referred to as a cell collecting apparatus) will be described with reference to FIG. 13.

The cell collecting apparatus is different from the cell collecting apparatus 1 according to the first embodiment in that the cell collecting apparatus includes the culturing device 400 illustrated in FIG. 13 instead of the culturing device 100. The cell collecting apparatus is similar to the cell collecting apparatus 1 in that the control device 10 is included.

As illustrated in FIG. 13, the culturing device 400 is different from the culturing device 100 in that it includes a microscope 170 instead of the camera 160 and that it includes a retroreflective member 180 attached to the culture vessel 110. The microscope 170 is an imaging device that captures an image of the inside of the culture vessel 110 from the outside of the culture vessel 110, and is arranged such that a tip of the objective lens 171 faces the side surface of the culture vessel 110. Note that the microscope 170 is a phase contrast microscope that images an object by a phase contrast observation method. However, the microscope 170 is not limited to the phase contrast microscope, and may image an object by using a bright field observation method, for example.

The retroreflective member 180 is used to cancel a lens effect that the light emitted from the microscope 170 receives on the side surface of the culture vessel 110. The retroreflective member 180 has an array in which a large number of minute reflective elements 181 are arranged in the horizontal direction. The reflective element 181 is, for example, a prism or a spherical glass bead. The retroreflective member 180 reflects the incident light by the reflective element 181 so as to direct the light to travel in the opposite direction in the same optical path as that at the time of incidence.

When the inside of the culture vessel 110 is imaged from the outside of the culture vessel 110, the image quality generally depends on the shape of the culture vessel 110. However, by using the microscope 170 together with the retroreflective member 180, as described in literatures such as WO 2019/163167 A, for example, the lens effect generated on the side surface of the culture vessel 110 can be canceled. Therefore, the target cell in the culture vessel 110 can be imaged from the outside of the culture vessel 110 with stable performance.

Also with the cell collecting apparatus according to the present embodiment, by executing the cell collecting process illustrated in FIG. 4, it is possible to automatically select and collect the target cell from a plurality of objects suspended in the liquid medium, similarly to the cell collecting apparatus according to the first embodiment to the third embodiment. This embodiment is similar to the above embodiment in that an arbitrary cell can be collected and that only the target cell can be collected.

### Fifth Embodiment

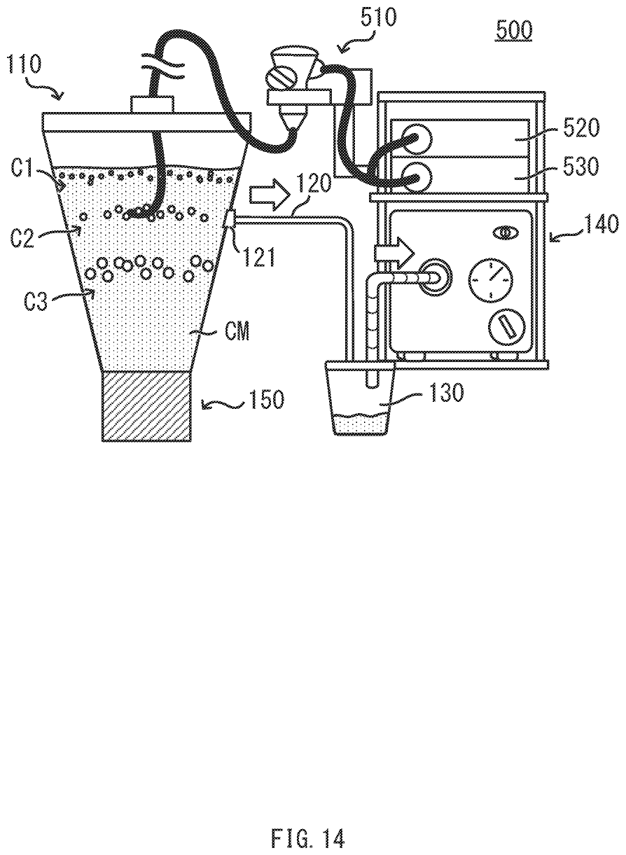

FIG. 14 is a diagram illustrating a configuration of a culturing device 500 according to the present embodiment. Hereinafter, the configuration of the cell collecting apparatus according to the present embodiment (hereinafter, simply referred to as a cell collecting apparatus) will be described with reference to FIG. 14.

The cell collecting apparatus is different from the cell collecting apparatus 1 according to the first embodiment in that the cell collecting apparatus includes the culturing device 500 illustrated in FIG. 14 instead of the culturing device 100. The cell collecting apparatus is similar to the cell collecting apparatus 1 in that the control device 10 is included.

As illustrated in FIG. 14, the culturing device 500 is different from the culturing device 100 in that it includes an endoscope 510, a video processor 520, and an illumination device 530, which are an example of an imaging device that images the inside of the culture vessel 110 from the inside of the culture vessel 110, instead of the camera 160.

The culturing device 500 images the target cell in the culture vessel 110 in a state where a distal end portion of the endoscope 510 is inserted into the liquid medium CM. More specifically, the culturing device 500 emits illumination light generated by the illumination device 530 from the distal end portion of the endoscope 510 toward the inside of the liquid medium CM to illuminate a region in the liquid medium CM. Furthermore, the culturing device 500 captures light from the region illuminated with the illumination light into the endoscope 510, converts the light captured in the endoscope 510 into an imaging signal by an imaging element, and further applies predetermined signal processing on the signal by using the video processor 520. With this processing, the culturing device 500 acquires an image of the inside of the liquid medium CM.

Also with the cell collecting apparatus according to the present embodiment, by executing the cell collecting process illustrated in FIG. 4, it is possible to automatically select and collect the target cell from a plurality of objects suspended in the liquid medium, similarly to the cell collecting apparatus according to the first embodiment to the fourth embodiment. This embodiment is similar to the above embodiment in that an arbitrary cell can be collected and that only the target cell can be collected.

### Sixth Embodiment

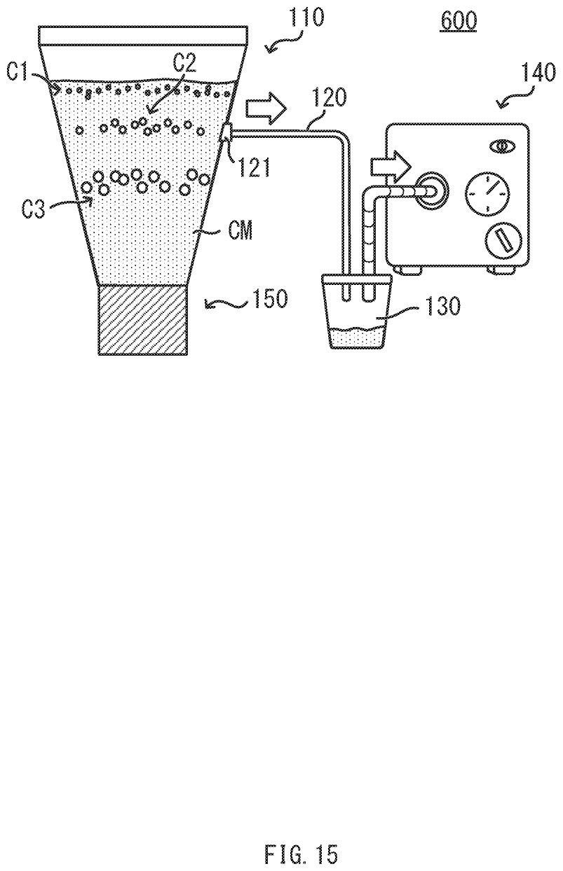

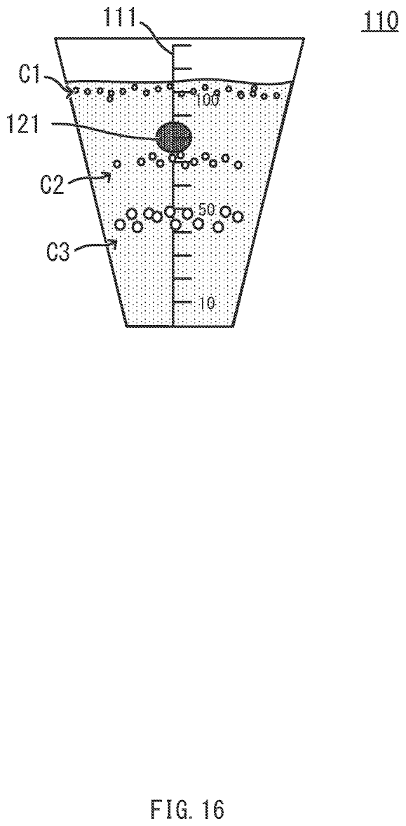

FIG. 15 is a diagram illustrating a configuration of a culturing device 600 according to the present embodiment. FIG. 16 is a diagram illustrating a method of confirming a suspension position of a cell. Hereinafter, the configuration of the cell collecting apparatus according to the present embodiment (hereinafter, simply referred to as a cell collecting apparatus) will be described with reference to FIGS. 15 and 16.

The cell collecting apparatus is different from the cell collecting apparatus 1 according to the first embodiment in that the cell collecting apparatus includes the culturing device 600 illustrated in FIG. 15 instead of the culturing device 100. The cell collecting apparatus is similar to the cell collecting apparatus 1 in that the control device 10 is included. As illustrated in FIG. 15, the culturing device 600 is different from the culturing device 100 in that the camera 160 is not included.

In the first to fifth embodiments, the cell collecting apparatus acquires information regarding a target cell such as the size and suspension height of the target cell from the image acquired by the imaging device. In contrast, in the present embodiment, since the cell collecting apparatus has no imaging device, the cell collecting apparatus acquires the information regarding a target cell from the user instead of acquiring the information regarding a target cell from the image.

In the present embodiment, when the user inputs information regarding a target cell to the control device 10 using the input device 13, the input device 13 receives the input information regarding the target cell, and then, the control device 10 controls the culturing device 600 so that the suspension height approaches the collection height based on the input information received by the input device 13. Note that the input information regarding the target cell input by the user desirably includes at least one of size information regarding the target cell (for example, the diameter of the target cell), position information regarding the target cell (for example, the suspension height of the target cell), and distance information between the suspension height and the collection height.

As illustrated in FIG. 16, the culture vessel 110 may be provided with a scale 111, and the user may read the suspension height of the target cell or the distance between the suspension height and the collection height (height of the inlet 121) by using the scale 111 provided on the culture vessel 110.

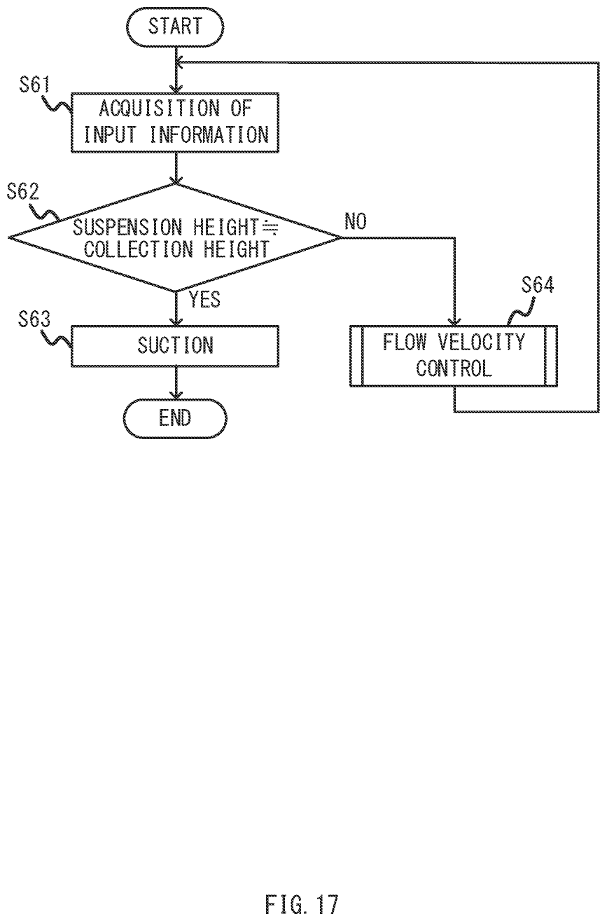

FIG. 17 is a flowchart illustrating an example of the cell collecting process according to the present embodiment. Hereinafter, a cell collecting method implemented by using the cell collecting apparatus according to the present embodiment will be specifically described with reference to FIG. 17.

The cell collecting process illustrated in FIG. 17 is started in accordance with a user's start instruction or a set schedule upon execution of the cell collecting program stored in the storage device 12 by the processor 11 of the control device 10. Note that the cell collecting process illustrated in FIG. 17 is different from the cell collecting process illustrated in FIG. 4 in that the input information described above is acquired in step S61 instead of image acquisition and object detection.

Accordingly, the control device 10 may control the flow velocity of the upward flow so that the suspension height approaches the collection height in step S64 based on the input information acquired in step S61. At that time, the control device 10 may estimate the sedimentation velocity of the target cell based on the input information, and may control the flow velocity of the upward flow in accordance with the estimated sedimentation velocity. Note that the estimation of the sedimentation velocity may be performed by using size information regarding the target cell included in the input information or using position information regarding the target cell included in the input information. The control device 10 may bring the suspension height close to the collection height by feedback control. For example, the suspension height and the collection height may be compared with each other using the positional information regarding the target cell included in the input information, or the flow velocity of the upward flow may be controlled according to the comparison result.

According to the cell collecting apparatus of the present embodiment, by executing the cell collecting process illustrated in FIG. 17, the target cell can be selectively collected from the plurality of objects suspended in the liquid medium only by inputting the input information related to the target cell by the user. This device is similar to that in the above-described embodiment in that an arbitrary cell can be collected and that only the target cell can be collected.

### Seventh Embodiment

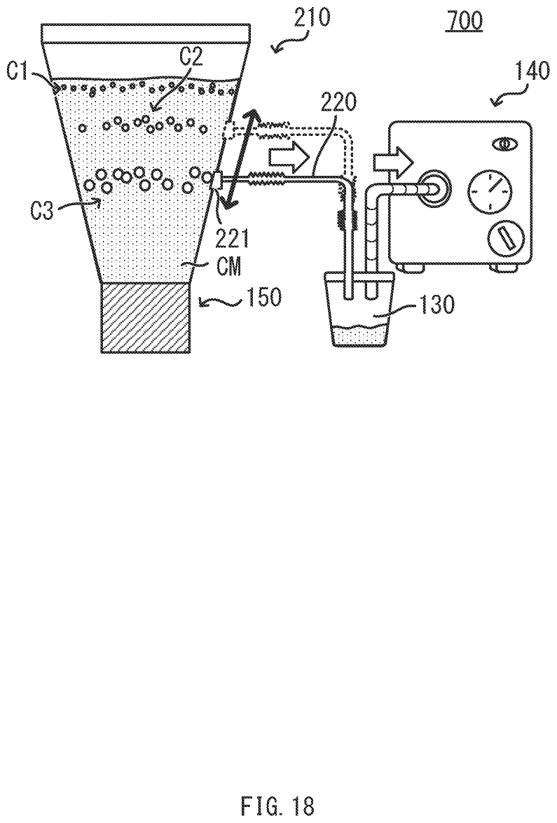

FIG. 18 is a diagram illustrating a configuration of a culturing device 700 according to the present embodiment. Hereinafter, the configuration of the cell collecting apparatus according to the present embodiment (hereinafter, simply referred to as a cell collecting apparatus) will be described with reference to FIG. 18.

The cell collecting apparatus is different from the cell collecting apparatus according to the second embodiment in that the cell collecting apparatus includes the culturing device 700 illustrated in FIG. 18 instead of the culturing device 200. The cell collecting apparatus is similar to the cell collecting apparatus according to the second embodiment in that the control device 10 is included. As illustrated in FIG. 18, the culturing device 700 is different from the culturing device 200 in that the camera 160 is not included.

Similarly to the sixth embodiment, the cell collecting apparatus in the present embodiment does not include the imaging device, and thus acquires information regarding the target cell from the user instead of acquiring the information regarding the target cell from the image. Furthermore, when the user inputs information regarding a target cell to the control device 10 using the input device 13, the input device 13 receives the input information regarding the target cell, and then, the control device 10 controls the culturing device 700 so that the suspension height approaches the collection height based on the input information received by the input device 13.

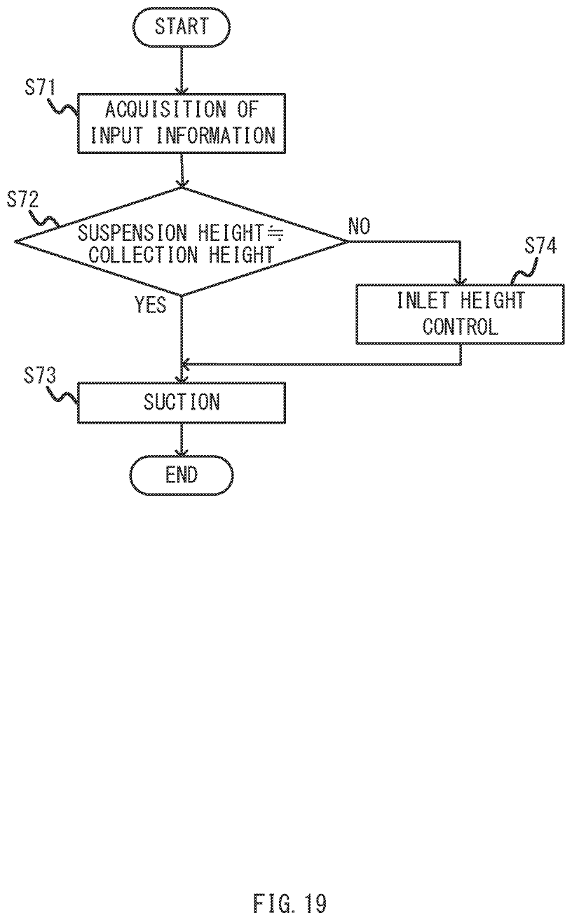

FIG. 19 is a flowchart illustrating an example of the cell collecting process according to the present embodiment. Hereinafter, a cell collecting method implemented by using the cell collecting apparatus according to the present embodiment will be specifically described with reference to FIG. 19.

The cell collecting process illustrated in FIG. 19 is started in accordance with a user's start instruction or a set schedule upon execution of the cell collecting program stored in the storage device 12 by the processor 11 of the control device 10. Note that the cell collecting process illustrated in FIG. 19 is different from the cell collecting process illustrated in FIG. 10 in that the input information described above is acquired in step S71 instead of image acquisition and object detection.

Therefore, the control device 10 may control the slider 222 (refer to FIG. 9) being the height adjuster so that the suspension height approaches the collection height in step S74 based on the input information acquired in step S71. More specifically, the control device 10 may control the slider 222 so that the slider 222 moves the inlet 221 in a direction of approaching the suspension height in step S74.

According to the cell collecting apparatus of the present embodiment, by executing the cell collecting process illustrated in FIG. 19, the target cell can be selectively collected from the plurality of objects suspended in the liquid medium only by inputting the input information related to the target cell by the user, similarly to the cell collecting apparatus according to the sixth embodiment. This embodiment is similar to the above embodiment in that an arbitrary cell can be collected and that only the target cell can be collected.

Also in the present embodiment, the suspension height may be brought close to the collection height by adjusting both the suspension height and the collection height. This point is similar to the second embodiment and the third embodiment.

### Eighth Embodiment

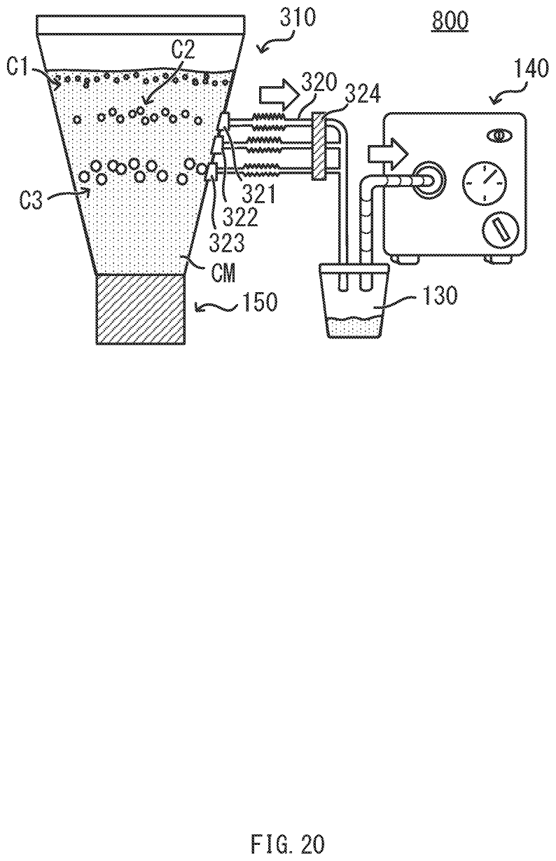

FIG. 20 is a diagram illustrating a configuration of a culturing device 800 according to the present embodiment. Hereinafter, the configuration of the cell collecting apparatus according to the present embodiment (hereinafter, simply referred to as a cell collecting apparatus) will be described with reference to FIG. 20.

The cell collecting apparatus is different from the cell collecting apparatus according to the third embodiment in that the cell collecting apparatus includes the culturing device 800 illustrated in FIG. 20 instead of the culturing device 300. The cell collecting apparatus is similar to the cell collecting apparatus according to the third embodiment in that the control device 10 is included. As illustrated in FIG. 20, the culturing device 800 is different from the culturing device 300 in that the camera 160 is not included.

Similarly to the seventh embodiment, the cell collecting apparatus in the present embodiment does not include the imaging device, and thus acquires information regarding the target cell from the user instead of acquiring the information regarding the target cell from the image. Furthermore, when the user inputs information regarding a target cell to the control device 10 using the input device 13, the input device 13 receives the input information regarding the target cell, and then, the control device 10 controls the culturing device 800 so that the suspension height approaches the collection height based on the input information received by the input device 13.

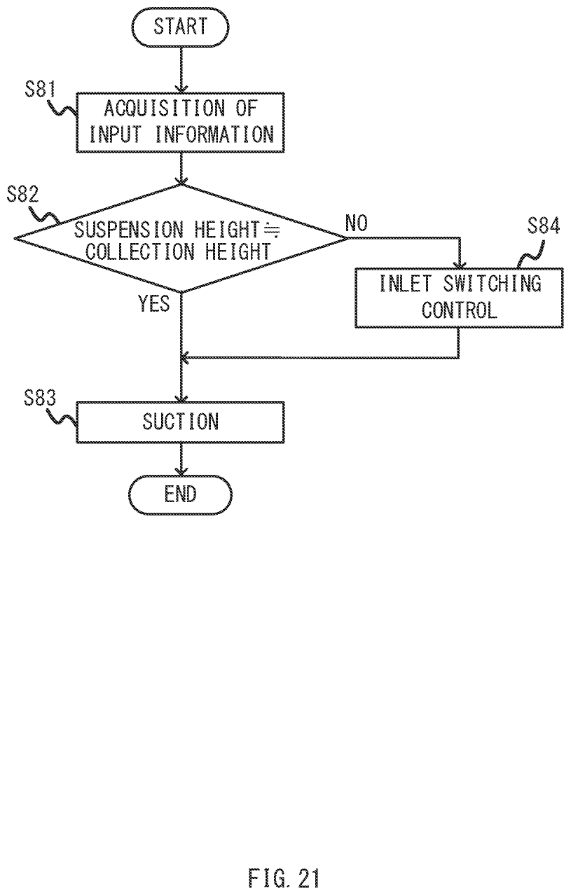

FIG. 21 is a flowchart illustrating an example of the cell collecting process according to the present embodiment. Hereinafter, a cell collecting method implemented by using the cell collecting apparatus according to the present embodiment will be specifically described with reference to FIG. 21.

The cell collecting process illustrated in FIG. 21 is started in accordance with a user's start instruction or a set schedule upon execution of the cell collecting program stored in the storage device 12 by the processor 11 of the control device 10. Note that the cell collecting process illustrated in FIG. 21 is different from the cell collecting process illustrated in FIG. 12 in that the input information described above is acquired in step S81 instead of image acquisition and object detection.

Therefore, the control device 10 may control the switching valve 324 being a height adjuster so that the suspension height approaches the collection height in step S84 based on the input information acquired in step S81. More specifically, the switching valve 324 may control the switching valve so that the switching valve 324 switches the inlet to be used for cell collection to the inlet closest to the suspension height among the plurality of inlets in step S84.

According to the cell collecting apparatus of the present embodiment, by executing the cell collecting process illustrated in FIG. 21, the target cell can be selectively collected from the plurality of objects suspended in the liquid medium only by inputting the input information related to the target cell by the user, similarly to the cell collecting apparatus according to the sixth embodiment and the seventh embodiment. This embodiment is similar to the above embodiment in that an arbitrary cell can be collected and that only the target cell can be collected.

Also in the present embodiment, the suspension height may be brought close to the collection height by adjusting both the suspension height and the collection height. This point is similar to the second embodiment, the third embodiment, and the seventh embodiment.

The above-described embodiments illustrate specific examples in order to facilitate understanding of the invention, and the present invention is not limited to these embodiments. Variations obtained by modifying the above-described embodiments and alternatives to the above-described embodiments can be included. That is, in each embodiment, the constituents can be modified without departing from the spirit and scope thereof. In addition, a new embodiment can be implemented by appropriately combining a plurality of constituents disclosed in one or more embodiments. In addition, some constituents may be deleted from the constituents illustrated in the respective embodiments, or some components may be added to the constituents illustrated in the embodiments. Furthermore, the processing procedures described in each embodiment may be performed in a different order as long as there is no contradiction. That is, the cell collecting apparatus, the cell collecting method, and the computer-readable medium of the present invention can be variously modified and altered without departing from the claims.

Although the embodiment described above is an example in which a cell is collected when the control device 10 has determined that the suspension height and the collection height are sufficiently close to each other, it is also allowable to confirm whether the collected cell is the target cell before starting full-scale cell collection. For example, it is also allowable to collect a small amount of cells using a collecting nozzle and analyze the collected cells after the control device 10 has determined that the suspension height and the collection height are sufficiently close to each other. An imaging device may be used for the analysis, and an image obtained by imaging the collected cell may be analyzed. Full-scale cell collection may be performed after the collected cell is confirmed to be the target cell based on the analysis result. This makes it possible to collect the target cell more reliably.

Although the embodiment described above is an example in which a cell is collected when the control device 10 has determined that the suspension height and the collection height are sufficiently close, the cell collecting apparatus may include a structure for avoiding cells different from the target cell from being sucked into the collecting channel at the time of collection, and may activate the structure before starting collection. For example, the culture vessel may include a movable structure that partitions the inside of the vessel, and it is allowable to move, before starting collection, the movable structure to the position between a height at which the target cell is suspended (≈collection height) and a height at which cells other than the target cell are suspended. This can reduce the possibility that cells other than the target cell are unintentionally sucked into the collecting channel at the time of collection.

Although the above-described embodiment is an example in which the cells or the cell mass are suspended by the upward flow, the cells or the cell mass may be suspended together with a carrier such as microbeads, in a state of being adhered to the carrier. That is, any culturing device may be used as long as the device can culture cells in a suspended state, and thus is not limited to the device for general suspension culture but the device for culturing cells by carrier suspension culture may be used.

Although the above-described embodiment is an example of estimating the sedimentation velocity using the diameter of the cell as the only variation factor, other information such as the density of the cell may be used for estimating the sedimentation velocity. The density of the target cell may be acquired from the server 3 in which a database is constructed in advance, for example, by the control device 10 transmitting information for identifying the target cell to the server 3.

In the present specification, “A approaches B” means all of (1) A approaches B as a result of movement of A, (2) A approaches B as a result of movement of B, and (3) A approaches B as a result of movement of both A and B. That is, the expression “A approaches B” may be used for the state change to shorten the distance between A and B, and it can be paraphrased as “A and B approach (each other)”.

What is claimed is:

1. A cell collecting apparatus comprising:
a culturing device including a culture vessel and a collecting channel, the culturing device configured to culture cells in a state where the cells are suspended in a liquid medium stored in the culture vessel; and
a control device configured to control the culturing device such that a suspension height, which is a height at which a target cell to be collected among the cells is suspended in the liquid medium, approaches a collection height, which is a height of an inlet of the collecting channel, the inlet configured to be used for collecting the cell from inside the culture vessel.

2. The cell collecting apparatus according to claim 1, wherein
the culturing device further includes an imaging device configured to image the target cell, and
the control device controls the culturing device such that the suspension height approaches the collection height based on a result of object detection on the image acquired by the imaging device.

3. The cell collecting apparatus according to claim 2, wherein
the culturing device further includes a suspension device configured to generate an upward flow in the liquid medium to allow the cells to be suspended, and
the control device controls a flow velocity of the upward flow such that the suspension height approaches the collection height based on a result of the object detection.

4. The cell collecting apparatus according to claim 3, wherein
the control device is configured to:
estimate a sedimentation velocity of the target cell based on a result of the object detection; and
control the flow velocity of the upward flow in accordance with the estimated sedimentation velocity.

5. The cell collecting apparatus according to claim 2, wherein
the result of the object detection includes size information regarding the target cell or position information regarding the target cell.

6. The cell collecting apparatus according to claim 2, wherein
the culturing device further includes a height adjuster configured to adjust the collection height, and
the control device controls the height adjuster such that the suspension height approaches the collection height based on a result of the object detection.

7. The cell collecting apparatus according to claim 6, wherein
the height adjuster includes a structure configured to move the inlet in a height direction, and
the control device controls the height adjuster so that the height adjuster moves the inlet in a direction of approaching the suspension height based on the result of the object detection.

8. The cell collecting apparatus according to claim 6, wherein
the collecting channel includes a plurality of inlets having mutually different heights and facing the culture vessel,
the height adjuster has a structure of switching an inlet to be used for cell collection among the plurality of inlets, and
the control device controls the height adjuster such that the height adjuster switches the inlet to be used for cell collection to an inlet closest to the suspension height among the plurality of inlets based on the result of the object detection.

9. The cell collecting apparatus according to claim 2, wherein
the culturing device further includes:
a suspension device configured to generate an upward flow in the liquid medium to allow the cells to be suspended; and
a height adjuster configured to adjust the collection height, and
the control device controls a flow velocity of the upward flow and the height adjuster such that the suspension height approaches the collection height based on a result of the object detection.

10. The cell collecting apparatus according to claim 1, wherein
the control device
includes an input device configured to receive input information related to information regarding the target cell, and
controls the culturing device such that the suspension height approaches the collection height based on the input information received by the input device.

11. The cell collecting apparatus according to claim 10, wherein
the culturing device further includes a suspension device configured to generate an upward flow in the liquid medium to allow the cells to be suspended, and
the control device controls a flow velocity of the upward flow such that the suspension height approaches the collection height based on the input information.

12. The cell collecting apparatus according to claim 11, wherein
the control device is configured to:
estimate a sedimentation velocity of the target cell based on the input information; and
control the flow velocity of the upward flow in accordance with the estimated sedimentation velocity.

13. The cell collecting apparatus according to claim 10, wherein
the input information includes at least one of size information regarding the target cell, position information regarding the target cell, and distance information between the suspension height and the collection height.

14. The cell collecting apparatus according to claim 10, wherein
the culturing device further includes a height adjuster configured to adjust the collection height, and
the control device controls the height adjuster such that the suspension height approaches the collection height based on the input information.

15. The cell collecting apparatus according to claim 14, wherein
the height adjuster includes a structure configured to move the inlet in a height direction, and
the control device controls the height adjuster so that the height adjuster moves the inlet in a direction of approaching the suspension height based on the input information.

16. The cell collecting apparatus according to claim 14, wherein
the collecting channel includes a plurality of inlets facing the culture vessel,
the height adjuster has a structure of switching an inlet to be used for cell collection among the plurality of inlets, and
the control device controls the height adjuster such that the height adjuster switches the inlet to be used for cell collection to an inlet closest to the suspension height among the plurality of inlets based on the input information.

17. The cell collecting apparatus according to claim 10, wherein
the culturing device further includes:
a suspension device configured to generate an upward flow in the liquid medium to allow the cells to be suspended; and
a height adjuster configured to adjust the collection height, and
the control device controls a flow velocity of the upward flow and the height adjuster such that the suspension height approaches the collection height based on the input information.

18. The cell collecting apparatus according to claim 1, wherein
the inlet of the collecting channel is formed on a side surface of the culture vessel.

19. A cell collecting method comprising:
culturing cells by using a culturing device in a state where the cells are suspended in a liquid medium stored in a culture vessel; and
controlling the culturing device by a control device such that a suspension height, which is a height at which a target cell to be collected among the cells is suspended in the liquid medium, approaches a collection height, which is a height of an inlet of a collecting channel, the inlet configured to be used for collecting the cell from inside the culture vessel.

20. A non-transitory computer-readable medium storing a program causing a computer to execute a process of
controlling a culturing device configured to culture cells in a state where the cells are suspended in a liquid medium stored in the culture vessel such that a suspension height, which is a height at which a target cell to be collected among the cells is suspended in the liquid medium, approaches a collection height, which is a height of an inlet of a collecting channel, the inlet configured to be used for collecting the cell from inside the culture vessel.
